(19) United States
(12) Patent Application Publication
(10) Pub. No.: US 2024/0255523 A1
(43) Pub. Date: Aug. 1, 2024

(54) CARTILAGE MODEL AS A SUBSTITUTE FOR ANIMAL MODELS AND METHODS FOR EVALUATING EFFECTIVENESS OR TOXICITY OF DRUGS USING THE SAME

(71) Applicant: KOREA RESEARCH INSTITUTE OF CHEMICAL TECHNOLOGY, Yuseong-gu Daejeon (KR)

(72) Inventors: Sun-Woong KANG, Yuseong-gu Daejeon (KR); Hye Eun SHIM, Yuseong-gu Daejeon (KR); Byoung-Seok LEE, Yuseong-gu Daejeon (KR); Ji Sek HAN, Seo-gu Daejeon (KR); Sumi JANG, Yuseong-gu Daejeon (KR)

(21) Appl. No.: 18/290,436

(22) PCT Filed: May 3, 2022
(86) PCT No.: PCT/KR2022/006348

§ 371 Date: Nov. 13, 2023

(30) Foreign Application Priority Data
May 13, 2021 (KR) 1020210062084

(51) Int. Cl. G01N 33/68; G01N 33/50

(52) U.S. Cl. CPC G01N 33/6887; G01N 33/5005; G01N 2333/78

(57) ABSTRACT

The present invention relates to a method for producing a cartilage model, comprising the step of simultaneously culturing a porous natural polymer support and a cartilage cell; a cartilage model produced by the method; and a method for testing the activity or toxicity of a test substance, comprising the step of treating the cartilage model with the test substance. A mature cartilage or aged cartilage model that can substitute an animal model may be produced using the method for producing the cartilage model according to the present invention, and the effectiveness and safety of the test substance may be accurately verified using the cartilage model thus produced, and thus, the cartilage model may be diversely utilized in the fields of new medicine development, disease research, and artificial organ development.

### TECHNICAL FIELD

The present invention relates to a method for producing a cartilage model, comprising the step of simultaneously culturing a porous natural polymer support and a cartilage cell; a cartilage model produced by the method; and a method for testing the activity or toxicity of a test substance, comprising the step of treating the cartilage model with the test substance.

### BACKGROUND ART

Cartilage is a tissue composed of cartilage cells and cartilage matrix, and cartilage has high elasticity to act as a shock absorber against a given force and has a very low coefficient of friction in articular cartilage to help joints move with little friction. Articular cartilage is composed of a large amount of cartilage matrix and cartilage cells, which are specially differentiated cells sparsely distributed between the cartilage matrices. Cartilage matrix includes water, proteoglycans and collagens as its main components, and also includes other proteins and glycoproteins. Articular cartilage is composed of the following four layers depending on the depth from the articular surface to the subchondral bone: superficial layer, middle layer, deep layer and calcified layer. When observed with an optical microscope, a wavy basophilic blue line is observed between the deep layer and the calcified layer and is referred to as a tide mark, and its number increases with age. Cartilage cells act to produce and maintain articular cartilage.

Diseases such as arthritis may be caused by damage or degenerative changes in the cartilage that protects joints like this, and animal testing in fracture or arthritis-induced animal models is essential to evaluate the effectiveness of arthritis treatment substances. Models for these animal tests are prepared by inducing arthritis through surgical operations, rib fractures such as artificial rib fractures, and artificial cartilage destruction, or by injecting bovine type II collagen. As an example, osteoarthritis-induced white rats or rabbits are used in animal testing, and the test substance is administered for 8 to 12 weeks, and then biochemical indicators such as hyaluronic acid, pentosidin and MMP9 in serum are confirmed, and histopathological observation and the like are conducted. However, these animal tests have limitations in that it is difficult to accurately evaluate drug effectiveness due to the short administration period of the test substance and that it is impossible to quickly evaluate drug efficacy.

Meanwhile, it has been reported that organoids, which are three-dimensional organ analogues, are cultured in materials such as Matrigel that mimic the extracellular matrix, and this three-dimensional culture technology has very little karyotypic change and no spontaneous cancerous process, unlike conventional two-dimensional cell culture methods. Thus, organ analogue culture is considered to be more advantageous for long-term preservation of in vivo characteristics than any other cell culture method proposed to date. Recently, three-dimensional organ-mimicking organ analogues are expected to be usefully applied in the field of precision medicine in the future because these can be essential disease-mimicking models not only for basic research in disease understanding but also for implementing patient-customized treatments.

Under these backgrounds, as a result of efforts to develop a model that can replace animal experiments, the present inventors have completed a cartilage model regenerated by tissue engineering techniques and a method for evaluating the in vitro effectiveness or safety of test substances using the same.

### DISCLOSURE

### Technical Problem

The present invention aims to solve the above-described problems and other problems associated therewith.

It is an exemplary object of the present invention to provide a method for producing a cartilage model, comprising the step of simultaneously culturing a porous natural polymer support and a cartilage cell.

It is another exemplary object of the present invention to provide a cartilage model produced by the method.

It is another exemplary object of the present invention to provide a method for testing the activity or toxicity of a test substance, comprising the step of treating the cartilage model with the test substance.

The technical problems to be achieved according to the technical idea of the invention disclosed in the present specification are not limited to the above-mentioned problems to be solved, and other problems not mentioned will be clearly understood by those skilled in the art from the description below.

### Technical Solution

These will be described in detail as follows. On the other hand, each description and embodiment disclosed in the present application may also be applied to each other description and embodiment. That is, all combinations of the various elements disclosed in this application fall within the scope of this application. In addition, it cannot be seen that the scope of the present application is limited by the specific descriptions described below.

As an aspect for achieving the above objects, the present invention provides a method for producing a cartilage model, comprising the step of simultaneously culturing a porous natural polymer support and a cartilage cell.

The term “natural polymer” in the present invention refers to a polymer material that is present in nature or produced by living organisms, and may include, but is not limited to, collagen, fibronectin, gelatin, chitosan, alginic acid, hyaluronic acid, and the like.

The term “cartilage” in the present invention refers to a tissue composed of cartilage cells and cartilage matrix, and cartilage is a body organ that has high elasticity to act as a shock absorber against a given force and has a very low coefficient of friction in articular cartilage to help joints move with little friction. Specifically, cartilage cells act to produce and maintain articular cartilage, and cell division occurs in chondroblasts, but once growth stops, cartilage cells no longer divide under normal circumstances.

The term “simultaneously” in the present invention means co-operation, including the same time zone, concurrently or simultaneously.

In the present invention, “cartilage model” is a cartilage tissue or cartilage-like structure obtained by culturing cartilage cells isolated from cartilage through tissue engineering, and such cartilage model may be a cartilage model in a growth stage, a cartilage model in a maturation stage, a cartilage model in an aging stage, or a cartilage model in a degeneration stage.

The term “cartilage model in a growth stage” in the present invention refers to a cartilage tissue that mimics the animal cartilage tissue in the growth stage by culturing cartilage cells isolated from cartilage through tissue engineering. In the present invention, the cartilage model in this growth stage may be produced by culturing a porous natural polymer support and a cartilage cell for less than 5 months, specifically for 2 to 3 months.

The cartilage model in the growth stage may be characterized by being a model in a stage in which the thickness of the outer layer of cartilage increases (FIG. 3). In addition, the cartilage model in the growth stage is positive for Ki67, which is a factor that identifies cell proliferation ability (FIG. 6), so that it is in the stage in which many cells proliferate, and can be characterized in that a negative reaction appears in TUNEL staining (FIG. 7) and staining appears uniformly without any areas of descoloration even in Safranin O staining (FIG. 4).

The term “cartilage model in a mature stage” in the present invention refers to a cartilage tissue that mimics the animal cartilage tissue in the mature stage by culturing cartilage cells isolated from cartilage through tissue engineering. In the present invention, the cartilage model in this mature stage may be produced by culturing a porous natural polymer support and a cartilage cell for 3 to 7 months, specifically for 5 to 7 months.

The cartilage model in the mature stage may be characterized by being a model in a stage in which the thickness of the outer layer of cartilage is maintained without increasing or decreasing (FIG. 3). The cartilage model in the mature stage may be characterized in that the cell shape maintains the lacuna structure well and GAGs are well formed (FIGS. 2A, 2B, and 4). In addition, the cartilage model in the mature stage may be characterized in that collagen type II is expressed, there are almost no proliferating cells in Ki67 staining, and apoptosis-positive cells begin to appear in TUNEL staining (FIG. 7).

The term “cartilage model in an aging stage” in the present invention refers to a cartilage tissue that mimics the animal cartilage tissue in the aging stage by culturing cartilage cells isolated from cartilage through tissue engineering. In the present invention, the cartilage model in this aging stage may be produced by culturing a porous natural polymer support and a cartilage cell for for 7 to 9 months.

The cartilage model in the aging stage may be characterized by being a model in a stage in which the thickness of the outer layer of cartilage decreases (FIG. 3). In addition, the cartilage model in the aging stage may be characterized in that the expression of collagen type II begins to decrease (FIG. 5), the number of apoptosis-positive cells increases in TUNEL staining, so that more cells are killed (FIG. 7), and the red layer decreases as the color of the outer layer of cartilage becomes lighter in Safranin O staining (FIG. 4).

The term “cartilage model in a degeneration stage” in the present invention refers to a cartilage tissue that mimics the animal cartilage tissue in the degeneration stage by culturing cartilage cells isolated from cartilage through tissue engineering. In the present invention, the cartilage model in this degeneration stage may be produced by culturing a porous natural polymer support and a cartilage cell for 10 months or more.

The model in the degeneration stage is in the stage in which most of the cells exhibit apoptosis, and may be characterized in that the epidermis of the outer layer is peeled off and the thickness of the outer layer is maintained in a reduced state. The cartilage model in the degeneration stage may be characterized in that the superficial cells are remarkably reduced and even peeled off (FIGS. 2A and 2B). In addition, it may be characterized in that collagen type I is expressed while the expression of collagen type II decreases (FIG. 5), and the red color of the outer layer of cartilage remarkably decreases in Safranin O staining (FIG. 4).

In addition, the present invention may comprise the step of selecting an cartilage model in a growth stage, a maturation stage, an aging stage or a degeneration stage, respectively, by confirming changes in the thickness of the outer layer in the cartilage model.

In addition, the present invention may further comprise the step of confirming the expression of collagen type I and collagen type II in the cultured cartilage model.

As an example, it may comprise the step of selecting, from the cultured cartilage models, a cartilage model in which collagen type I is hardly expressed but collagen type II is expressed, as a cartilage model in a growth stage, a maturation stage or an aging stage, and may comprise the step of selecting, from the cultured cartilage models, a cartilage model in which collagen type I is expressed but collagen type II is hardly expressed, as a cartilage model in a degeneration stage.

Additionally, it may further comprise the step of confirming the formation of lacuna or glycosaminoglycan (GAG) in the cultured cartilage model.

As an example, it may comprise the step of selecting, from the cultured cartilage models, a cartilage model in which the lacuna structure is maintained or the GAG is formed, as a cartilage model in a mature stage.

In addition, the present invention may further comprise the step of confirming the expression of Ki67 in the cultured cartilage model.

As an example, it may comprise the step of selecting, from the cultured cartilage models, a cartilage model in which Ki67 is expressed and cell proliferation is observed, as a cartilage model in a growth stage, and may comprise the step of selecting, from the cultured cartilage models, a cartilage model in which Ki67 is hardly expressed and cell proliferation is hardly observed, as a cartilage model in a mature stage.

In addition, the present invention may further comprise the step of confirming apoptosis of the cartilage cell in the cultured cartilage model through a TUNEL assay.

As an example, from the cultured cartilage models, it may comprise the step of selecting a cartilage model in which apoptosis of the cartilage cell is not observed, as a cartilage model in a growth stage; the step of selecting a cartilage model in which an apoptosis-positive cell begins to appear, as a cartilage model in a mature stage; the step of selecting a cartilage model in which an apoptosis-positive cell increases and cell death increases, as a cartilage model in an aging stage; and/or the step of selecting a cartilage model in which most of the cells exhibit an apoptotic state, as a cartilage model in a degeneration stage.

Some or all of the above-mentioned steps may be performed to produce cartilage models in appropriate growth, maturation, aging and degeneration stages.

As another aspect for achieving the above objects, the present invention provides a cartilage model manufactured by the method.

The cartilage model according to the present invention exhibits the characteristics of growth, maturation, aging or degeneration of cartilage tissue, so that it may be used as a substitute for animal models to evaluate the effects of repeated or sequential treatment of test substances.

As another aspect for achieving the above objects, the present invention provides the use of a cartilage model for confirming the activity or toxicity of a substance.

In the present invention, the substance includes chemical or biological substances and are applicable to the present invention without particular limitation.

As another aspect for achieving the above objects, the present invention provides a method for testing the activity or toxicity of a test substance, comprising the step of treating the cartilage model with the test substance.

The phrase “method for testing the activity or toxicity” in the present invention is not limited to the type, and may be an evaluation method based on cartilage components, proliferation of cartilage cells, cartilage cell death, tissue shape, activity of osteoblasts and osteoclasts, lesions of cartilage tissue, and the like, when the activity or toxicity is tested using a cartilage model.

In this method, the preparation of cartilage models in the growth stage, maturation stage, aging stage and degeneration stage is prepared with reference to the method for producing the cartilage model as mentioned above.

In the present invention, the activity of the test substance may be, but is not limited to, a measurement of drug metabolic activity or an assessment of drug interaction.

### Advantageous Effects

A mature cartilage or aged cartilage model that can substitute an animal model may be produced using the method for producing the cartilage model according to the present invention, and the effectiveness and safety of the test substance may be accurately verified using the cartilage model thus produced, and thus, the cartilage model may be diversely utilized in the fields of new medicine development, disease research, and artificial organ development.

However, the effects according to an embodiment of the technology disclosed in the present specification are not limited to those mentioned above, and other effects not mentioned will be clearly understood by those skilled in the art from the description below.

### DESCRIPTION OF DRAWINGS

In order to more fully understand the drawings cited in present specification, a brief description of each drawing is provided.

FIG. 1 shows the result of primary culture of cartilage cells isolated from rabbit cartilage.

FIGS. 2A and 2B are the results of tissue staining in cartilage models tissue cultured for each period of time.

FIG. 3 shows the results of measuring the thickness of the outer layer in cartilage models tissue cultured for each period of time.

FIG. 4 shows the results of Safranin O staining to confirm GAG content in cartilage models tissue cultured for each period of time.

FIG. 5 shows the results of comparing the expression of collagen type I and type II in cartilage models tissue cultured for each period of time.

FIG. 6 shows the results of immunohistostaining to identify Ki67 expression to confirm cell proliferation in cartilage models tissue cultured for each period of time.

FIG. 7 shows the results of the TUNEL assay to confirm degeneration in cartilage models tissue cultured for each period of time.

FIGS. 8A, 8B and 8C show the results of live/dead assay in cartilage models tissue cultured for each period of time.

### BEST MODE

Hereinafter, the present invention will be described in more detail through the following examples. However, these examples are only for illustrating the present invention, and the scope of the present invention is not limited to these examples only.

### Example 1: Production of Cartilage Model Using Porous Collagen Support

A three-dimensional culture system was constructed using a porous collagen support, and the three-dimensional culture system has an improved structure compared to that cultured in a commonly used two-dimensional environment, thereby allowing closer interaction with adjacent cells and better maintaining the three-dimensional shape.

### Example 1-1: Primary Culture of Cartilage Cells

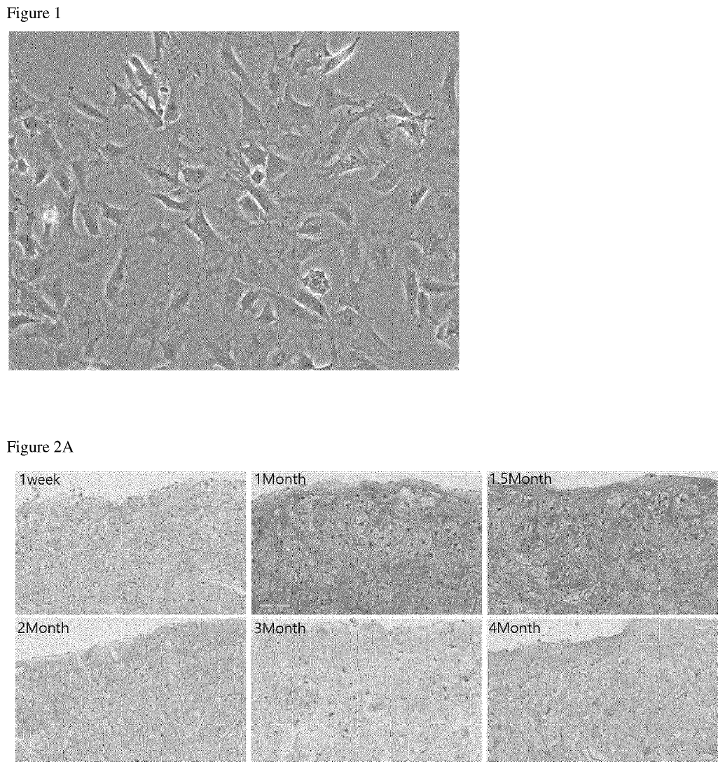

After the rabbit was euthanized, the hind leg bones were separated. The cartilage tissue present in the condyle of the rabbit femur was thinly peeled off using a sterilized blade. The fragmented cartilage tissue was minced with a blade in a clean Petri dish and cut into smaller pieces, and in this case, the cartilage tissue was used while sufficiently moistened with a buffer solution such as PBS. Finely shredded cartilage tissue was placed in DMEM medium containing 0.05% collagenase type II and cultured in an incubator under conditions at 37° C. and 5% CO₂ for 18 to 24 hours until the matrix was completely decomposed. After inactivating collagenase using culture medium containing fetal bovine serum (FBS), the cell suspension was filtered through a clean 50 ml conical tube using a 70 μm filter. Cartilage cells were obtained by centrifugation at 1200 rpm at room temperature for 10 minutes, and cultured in DMEM medium from 0 to 2 passages. Cartilage cells isolated from rabbit cartilage by primary culture are as shown in FIG. 1.

### Example 1-2: Preparation of Porous Collagen Support (Collagen Sponge)

A support was prepared by cutting a collagen sponge (Collacote) into 5×5×5 mm using a razor. Since it has a very soft nature due to the nature of the support, it was cut slowly without applying force with a razor to maintain the shape of the collagen sponge.

### Example 1-3: Culture of Cartilage Model

All culture medium from the primary cultured rabbit cartilage cells was removed by aspiration, and then washed twice with PBS, and treated with 0.25% trypsin-EDTA to remove the cartilage cells attached to the bottom of the culture dish. When the cells were suspended, trypsin-EDTA was inactivated with culture medium (DMEM) containing 10% FBS, and then collected in a fresh 50 ml conical tube and centrifuged at 1000 rpm for 3 minutes. The supernatant medium in the tube was removed by aspiration, and fresh medium was added to resuspend the cell pellet. 20 μl of the cell suspension was dispensed onto the cut collagen sponge and transferred to a 60 mm ultra-low attachment dish or Petri dish. The collagen sponge onto which the cell suspension was dispensed was cultured in an incubator at 37° C. and 5% CO₂ for 3 hours. During the culture, 10 μl of fresh culture medium was dispensed onto the collagen sponge at intervals of 30 minutes to provide the minimum amount of medium and time for the cells to attach to the sponge. The dish was filled with 8 ml or more of culture medium so that the collagen sponge with attached cells could be sufficiently submerged in the medium. It was confirmed through an optical microscope whether the cells were attached to the collagen sponge onto which the cell suspension was dispensed, and the cartilage model was completed by culturing while replacing the medium with fresh medium at intervals of 2 to 3 days. In this case, the cartilage model was produced by culturing within 2-3 months for the growth stage, 5 to 7 months for the maturation stage, 7 to 9 months for the aging stage, and 10 months or more for the degeneration stage.

### Example 2: Slide Imaging Method

Stained slides obtained through the staining method were observed under an optical microscope, and images of the slides were obtained using one of the following methods:
1) Partial images of the sample were obtained through a camera (DP74, Olympus) attached to an optical microscope; or
2) The entire slide was scanned using a 20× or 40× objective lens on a slide scanner (Aperio Scanscope XT, Vista) to convert the glass slide into a digital slide file, and then images were obtained at the desired location and magnification.

### Experimental Example 1: Observation of the Cartilage Model According to the Present Invention Through H&E Staining

The cartilage model produced in the same manner as in Example 1 above was fixed with 4% glutaraldehyde for one day or more. The processes of dehydration (70%, 80%, 90%, 95%, 100% ethanol), clearing (xylene) and paraffin infiltration were performed through an automatic tissue processor, and then paraffin blocks were prepared by embedding, sectioned to a thickness of 3-4 microns, placed on a slide glass, and dried at room temperature to prepare paraffin sections.

After these were left in a drying oven at 40° C. for 1 hour, the processes of deparaffinization (2 times xylene) and hydration (100%, 95%, 90%, 80%, 70% ethanol) were carried out step by step, and then transferred to water. These were stained by soaking in Harris' Hematoxylin solution for 10 minutes, and then placed in water for 10 minutes. The overstained Harris' Hematoxylin was washed in hydrochloric acid alcohol solution (mixed solution of 5 ml of 37% aqueous hydrochloric acid solution and 995 ml of 80% ethanol) for 30 seconds and transferred to water, and then placed in 0.5% ammonia solution for 30 seconds for bluing and transferred to water. These were stained with eosin for 3 minutes, and the processes of dehydration (95% and 100% ethanol, 3 times each) and clearing (2 times xylene) were carried out step by step, and then embedded and observed through an optical microscope.

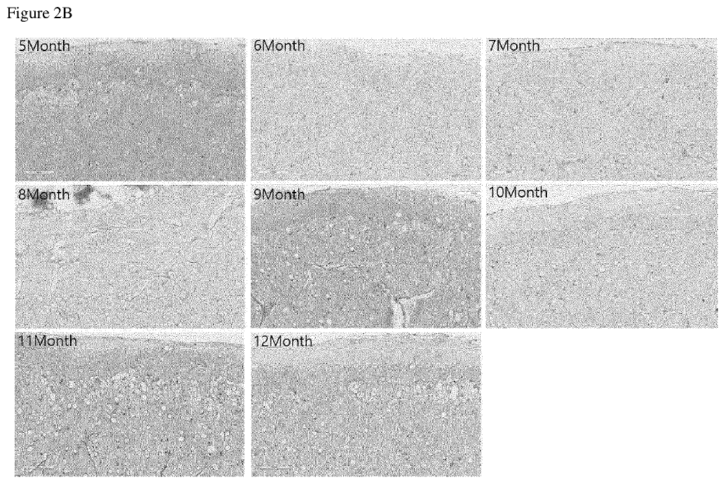

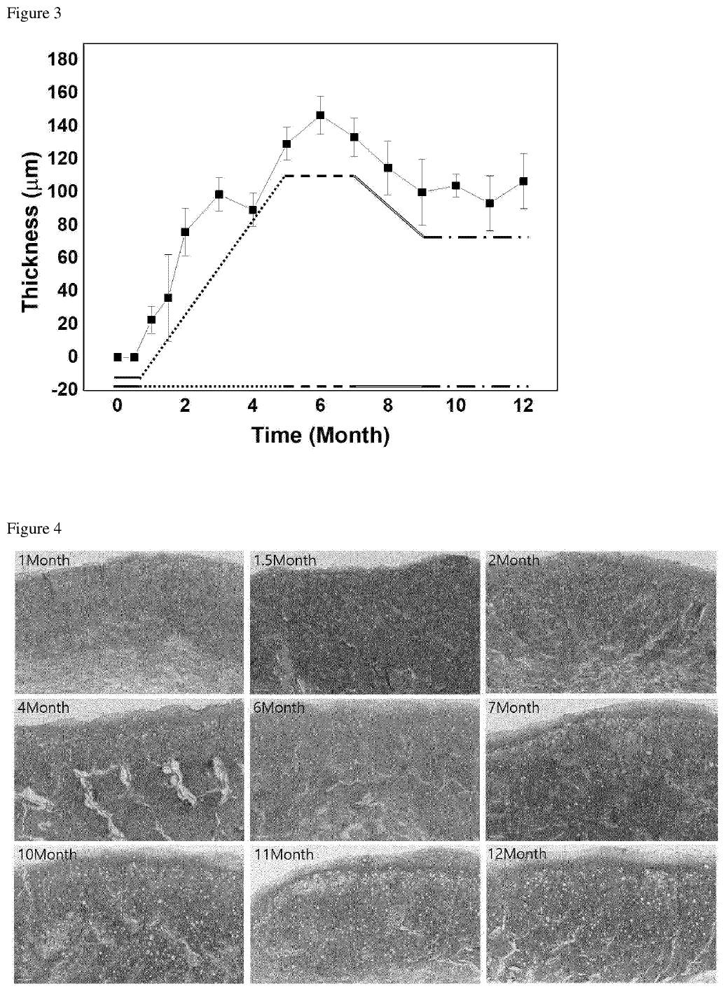

As a result of observing cartilage models produced for each period of time through H&E staining, it was confirmed that lacuna, which is a representative characteristic of cartilage cells, was generated, and it could be confirmed that the thickness of the outer layer changes over time. In particular, it could be confirmed that the thickness of the outer layer increases over time until 5 months of culture, and specifically, it could be confirmed that it increases to 100 to 120 um until 5 months. It could be confirmed that the thickness is maintained at 100 to 120 um for 5 to 7 months, and it could be confirmed that the thickness of the outer layer decreases to 60 to 80 um for 7 to 9 months and is maintained at 60 to 80 um for 9 months or more. That is, it could be confirmed that the thickness changes according to the growth stage, maturation stage, aging stage or degeneration stage in the cartilage model (FIGS. 2A, 2B and 3).

### Experimental Example 2: Confirmation of GAGs in the Cartilage Model According to the Present Invention Through Safranin O Staining

Paraffin sections of 3-4 microns attached to a slide glass were prepared in the same manner as in Experimental Example 1, and then the processes of deparaffinization (2 times xylene) and hydration (100%, 95%, 90%, 80%, 70% ethanol) were carried out step by step.

These were stained with Weigert's iron hematoxylin solution for 10 minutes, and then washed with running tap water for 10 minutes. Subsequently, these were stained with 0.05% Fast Green (FCF) aqueous solution for 5 minutes, and washed with 1% acetic acid aqueous solution for 10-15 seconds. Staining was performed with 0.1% Safranin O aqueous solution for 5 minutes, and the processes of dehydration (70%, 80%, 90%, 95%, 100% ethanol) and clearing (2 times xylene) were performed step by step, and then embedded and observed through an optical microscope.

As a result of confirming the presence of GAG in the cartilage model according to the present invention through Safranin O staining, it could be confirmed that GAG, which is a major component of cartilage, is produced for each period of time (FIG. 4). It could be confirmed that there was no change in the central part of the tissue, and the color of the outer layer of the epidermis became lighter after 10 months.

### Experimental Example 3: Comparison of the Expression of Collagen and Ki67 in the Cartilage Model According to the Present Invention Through Immunostaining

Paraffin sections of 3-4 microns attached to a slide glass were prepared in the same manner as in Experimental Example 1, and then the paraffin sections were placed in a slide warmer set at 48.5° C. for 40-50 minutes and attached to the slide glass. A slide glass labeled with a barcode that can be recognized by the device is placed on the slide tray of a slide automated immunostaining device (DISCOVERY XT, Roche Ventana), and the processes of paraffin removal, hydration and antigen retrieval (citrate buffer, CC2, 28 minutes) were carried out through the automated immunostaining device. In the primary antibody step, the tray was opened, and 100 ul of primary antibody diluted in 0.01% Tween 20 was added directly to each slide, wherein each antibody and ratio used are as follows: Collagen II Antibody (5B20.5), NB600-844, Novus, 1:50; Collagen I alpha 1 Antibody (COL-1), NB600-450, Novus, 1:100; beta-Galactosidase-1 Antibody (OTI1C9), NBP2-45731, Novus, 1:100. The program of the automated immunostaining device was continuously performed to proceed with primary antibody reaction (2 hours), secondary antibody reaction (UltraMap anti-Ms HRP, 20 minutes), DAB color development (ChromoMap DAB kit), and counter staining (Hematoxylin & Bluing reagent, 12 minutes). The slide with completed staining was removed from the automated immunostaining device, and then carefully washed with tap water containing detergent to remove the liquid coverslip (REF 650-010) component from Ventana. Subsequently, the processes of dehydration (70%, 80%, 90%, 95%, 100% ethanol) and clearing (2 times xylene) were performed step by step, and then embedded and observed through an optical microscope.

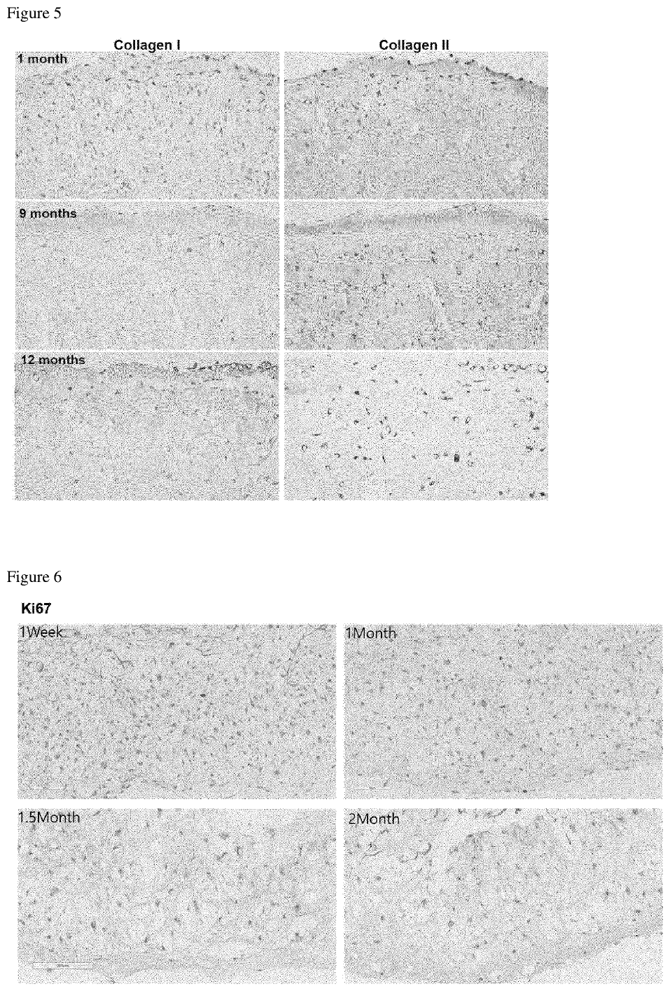

As a result of comparing the expression of collagen type I and type II in the cartilage model according to the present invention by the above immunostaining method, it could be confirmed that at 1 month and 9 months, collagen type I is not expressed well but collagen type II is expressed well; however, it could be confirmed that this was reversed after 9 months, and the expression of collagen type I increases but collagen type II was not expressed well (FIG. 5).

In addition, as a result of confirming the expression of Ki67, which is a factor that can identify cell proliferation ability, for each culture period of time in the cartilage model according to the present invention by the above immunostaining method, it could be confirmed that most of the cells proliferates up to 1.5 months. It could be confirmed that the number of proliferating cells decreases rapidly starting from 2 months.

### Experimental Example 4: Confirmation of Cell Death in the Cartilage Model According to the Present Invention Through TUNEL Assay

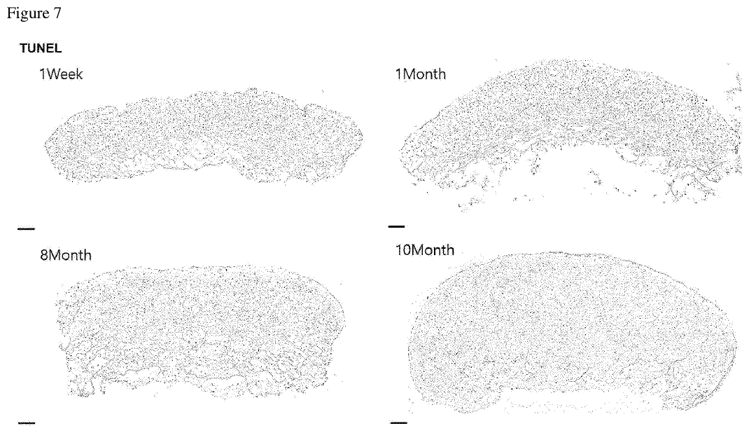

The TUNEL assay is a method that can identify dead cells, and as a result of confirming the cartilage model according to the present invention for each period of time through the TUNEL assay, brown stained areas could be confirmed in several places through tissue staining from 8 months onwards, and this result confirm that cell death, i.e., aging, of cartilage cells occurs (FIG. 7).

### Experimental Example 5: Confirmation of Cell Body Status in the Cartilage Model According to the Present Invention Through Live/Dead Assay

Some of the cartilage models prepared in the same manner as in Example 1 above were confirmed for the state of cell bodies being cultured by staining dead cells (red-fluorescent ethidium homodimer-1) and living cells (green-fluorescent calcein-AM) with live/dead fluorescent staining (Invitrogen™, LIVE/DEAD™ Viability/Cytotoxicity Kit).

Component A (4 mM calcein AM stock) and Component B (2 mM EthD-1 stock solution) stored at −20° C. were taken out and placed at room temperature. A 5× live-dead assay mixture was prepared at 50 ul/sample according to the number of samples, and 2.5 ul of Component A and 10 ul of Component B per 1 ml of fresh culture medium were added to contain 10 uM calcein AM and 20 uM EthD-1. In an 8-well chamber slide (ibidi, μ-Slide 8 Well), the same number of chambers as the number of samples to be analyzed were filled with 200 ul of fresh culture medium, respectively. The cartilage model being cultured is transferred to the plate so that each chamber contains one cartilage model. 50 ul of the prepared 5× Mixture was added to each chamber and mixed with a pipette, and then incubated at 37° C. for 10 minutes. Finally, the culture medium containing 2 uM calcein AM and 4 uM EthD-1 was prepared. In a confocal microscope, the images were confirmed with Alexa Fluor 488 settings for live cells (calcein staining) and Alexa Fluor 594 settings for dead cells (EthD-1 staining), and the transmitted light detector images for identifying the approximate shape were also confirmed. To obtain an overall image of cells located on the surface of the cartilage model that can be observed through the laser of a confocal microscope, a Z-stack image was taken, and then projection was performed.

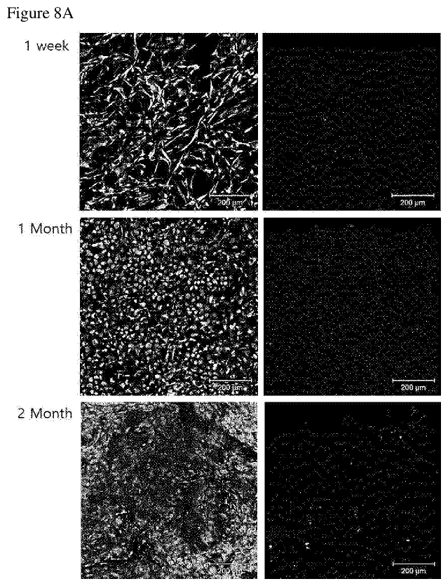

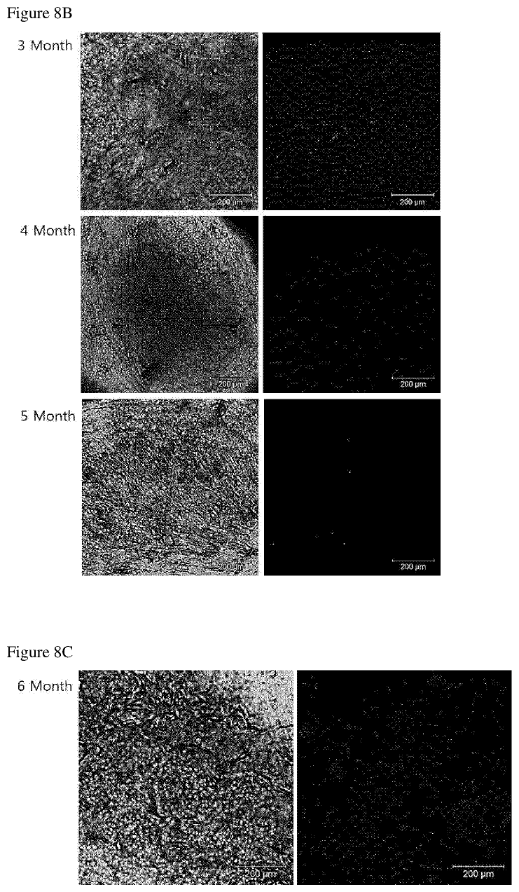

As a result, as shown in FIGS. 8A to 8C, it can be seen that most of the cells show green fluorescence, and thus are being cultured in a live state, and it can be seen that the density of cells increases up to 1 week, 1 month and 2 months. This is because the population increased through cell proliferation.

1. A method for producing a cartilage model, comprising the step of simultaneously culturing a porous natural polymer support and a cartilage cell.

2. The method according to claim 1, wherein the natural polymer is one or more selected from the group consisting of collagen, fibronectin, gelatin, chitosan, alginic acid, and hyaluronic acid.

3. The method according to claim 1, characterized in that the cartilage model is a cartilage model in a growth stage, a maturation stage, an aging stage or a degeneration stage.

4. The method according to claim 1, wherein the culturing is performed within 5 months, for 5 to 7 months, for 7 to 9 months, or for 10 months or more.

5. The method according to claim 1, characterized in that the method further comprises the step of confirming changes in the thickness of the outer layer in the cultured cartilage model.

6. The method according to claim 5, characterized in that the method comprises the step of selecting an cartilage model in a growth stage, a maturation stage, an aging stage or a degeneration stage by confirming changes in the thickness of the outer layer in the cartilage model.

7. The method according to claim 1, characterized in that the method further comprises the step of confirming the expression of collagen type I and collagen type II in the cultured cartilage model.

8. The method according to claim 7, characterized in that the method comprises the step of selecting, from the cultured cartilage models, a cartilage model in which collagen type I is hardly expressed but collagen type II is expressed, as a cartilage model in a growth stage, a maturation stage or an aging stage.

9. The method according to claim 7, characterized in that the method comprises the step of selecting, from the cultured cartilage models, a cartilage model in which collagen type I is expressed but collagen type II is hardly expressed, as a cartilage model in a degeneration stage.

10. The method according to claim 1, characterized in that the method further comprises the step of confirming the formation of lacuna or glycosaminoglycan (GAG) in the cultured cartilage model.

11. The method according to claim 10, characterized in that the method comprises the step of selecting, from the cultured cartilage models, a cartilage model in which the lacuna structure is maintained or the GAG is formed, as a cartilage model in a mature stage.

12. The method according to claim 1, characterized in that the method further comprises the step of confirming the expression of Ki67 in the cultured cartilage model.

13. The method according to claim 12, characterized in that the method comprises the step of selecting, from the cultured cartilage models, a cartilage model in which Ki67 is expressed and cell proliferation is observed, as a cartilage model in a growth stage.

14. The method according to claim 12, characterized in that the method comprises the step of selecting, from the cultured cartilage models, a cartilage model in which Ki67 is hardly expressed and cell proliferation is hardly observed, as a cartilage model in a mature stage.

15. The method according to claim 1, characterized in that the method further comprises the step of confirming apoptosis of the cartilage cell in the cultured cartilage model through a TUNEL assay.

16. The method according to claim 15, characterized in that the method comprises the step of selecting, from the cultured cartilage models, a cartilage model in which apoptosis of the cartilage cell is not observed, as a cartilage model in a growth stage.

17. The method according to claim 15, characterized in that the method comprises the step of selecting, from the cultured cartilage models, a cartilage model in which an apoptosis-positive cell begins to appear, as a cartilage model in a mature stage.

18. The method according to claim 15, characterized in that the method comprises the step of selecting, from the cultured cartilage models, a cartilage model in which an apoptosis-positive cell increases and cell death increases, as a cartilage model in an aging stage.

19. The method according to claim 15, characterized in that the method comprises the step of selecting, from the cultured cartilage models, a cartilage model in which most of the cells exhibit an apoptotic state, as a cartilage model in a degeneration stage.

20. A cartilage model produced by the method according to claim 1.

21. A method for testing the activity or toxicity of a test substance, comprising the step of treating the cartilage model according to claim 20 with the test substance.

22. The method according to claim 21, wherein the activity is a measurement of drug metabolic activity or an assessment of drug interaction.
